(19) United States
(12) Patent Application Publication
(10) Pub. No.: US 2026/0020781 A1
(43) Pub. Date: Jan. 22, 2026

(54) SYSTEM AND METHODS FOR COMPLETING AND SMOOTHING ANALYTE SENSOR SYSTEM SIGNALS

(71) Applicant: Dexcom, Inc., San Diego, CA (US)

(72) Inventors: Spencer Troy FRANK, San Diego, CA (US); Thibault Pierre GAUTIER, Dulles, VA (US); Jee Hye PARK, San Diego, CA (US); Justin Yi-Kai LEE, San Diego, CA (US); Enrique CAMPOS NÁÑEZ, Charlottesville, VA (US); Matthew Steven GERBER, Charlottesville, VA (US); Stephanie Grace MOORE, San Diego, CA (US)

(21) Appl. No.: 19/090,763

(22) Filed: Mar. 26, 2025

Related U.S. Application Data
(60) Provisional application No. 63/673,887, filed on Jul. 22, 2024.

(51) Int. Cl. A61B 5/145; A61B 5/00

(52) U.S. Cl. CPC A61B 5/14532; A61B 5/1451; A61B 5/7225; A61B 5/742

(57) ABSTRACT

The present disclosure relates to an analyte monitoring system for completing and smoothing analyte sensor signals. The system includes an analyte sensor system, a memory, and a processor. The processor detects a first gap in a time series of analyte sensor measurements from the analyte sensor system. The first gap is between a first data point in the time series and a second data point in the time series. The processor interpolates between the first data point and the second data point to determine a fill data point and adds the fill data point to the first gap. The processor determines an average of at least (i) a value of the first data point, (ii) the fill data point, and (iii) a third data point preceding the first data point in the time series and sets the value of the first data point to the determined average.

### CROSS-REFERENCE TO RELATED APPLICATIONS

This application claims priority to and benefit of U.S. Provisional Application No. 63/673,887, filed Jul. 22, 2024, which is hereby expressly incorporated by reference herein in its entirety as if fully set forth below and for all applicable purposes.

### BACKGROUND

Diabetes mellitus is a metabolic condition relating to the production or use of insulin by the body. Insulin is a hormone that allows the body to use glucose for energy, or store glucose as fat.

When a person cats a meal that contains carbohydrates, the digestive system absorbs nutrients, ultimately depositing glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), skin ulcers, and kidney damage. Hyperglycemia can also lead to acute problems, such as diabetic ketoacidosis—a state in which the body becomes excessively acidic due to the production of excess ketones, or body acids. The state of having lower than normal blood glucose levels is called “hypoglycemia.” Severe hypoglycemia can lead to damage of the heart muscle, neurocognitive dysfunction, and in certain cases, acute crises that can result in seizures or even death.

A patient living with diabetes can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Patients with diabetes can benefit from real-time diabetes management guidance, as determined based on a physiological state of the patient, in order to stay within a target glucose range and avoid physical complications. In certain cases, the physiological state of the patient is determined using monitoring systems that measure glucose levels, which inform the identification and/or prediction of adverse glycemic events, such as hyperglycemia and hypoglycemia, and the type of guidance provided to the patient.

For example, such monitoring systems may utilize a continuous glucose monitor (CGM) to measure a patient's glucose levels over time. The measured glucose levels may then be processed by the monitoring system to identify and/or predict adverse glycemic events, and/or to provide guidance to the patient for treatment and or actions to abate or prevent the occurrence of such adverse glycemic events. For example, trends, statistics, or other metrics may be derived from the glucose levels and used to identify and/or predict adverse glycemic events. Or, in certain cases, the glucose levels themselves may be used to identify and/or predict adverse glycemic events.

Even with the systems described above, however, the management of diabetes presents many challenges for patients, clinicians, and caregivers, as a confluence of various factors can impact a patient's glucose levels, thus affecting the accuracy of glycemic event prediction and the guidance provided by diagnostics systems.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a diagram conceptually illustrating an example continuous analyte monitoring system including an example continuous analyte sensor with sensor electronics and example display devices, in accordance with certain aspects of the present disclosure.

FIGS. 2A through 2F illustrate example operations for filling signal gaps in signal streams performed by the continuous analyte monitoring system of FIG. 1, in accordance with certain aspects of the present disclosure.

FIGS. 3A through 3C illustrate example operations for adjusting data points in signal streams performed by the continuous analyte monitoring system of FIG. 1, in accordance with certain aspects of the present disclosure.

FIG. 4 is a flowchart of an example method performed by the continuous analyte monitoring system of FIG. 1, in accordance with certain aspects of the present disclosure.

FIG. 5 illustrates an example computer system, in accordance with certain aspects of the disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In a continuous analyte monitoring system, a transcutaneous continuous analyte sensor that is inserted into the interstitial fluid is used to monitor a patient's analyte levels, thereby, providing analyte concentration measurements reflective of the physiological state of the patient. An analyte may be understood as any substance of interest that is to be measured or is being measured. Examples of such analytes include glucose, ketones, lactate, insulin, electrolytes, creatinine, as well as a number of other biomarkers including proteins, metabolites, and nucleic acids. The sensor electrodes of the transcutaneous continuous analyte sensor may interact with the desired analyte (e.g., through aptamers (single-stranded DNA or RNA molecules that bind to a specific analyte)). An analyte of particular interest may be referred to as a target analyte. For example, in a transcutaneous continuous glucose sensor, the target analyte may be glucose.

The concentration of the target analyte in the patient may cause the continuous analyte sensor to generate an electric signal (e.g., an electric current or voltage). The continuous analyte sensor converts the electric signal into a signal that includes a time series of data points, with each data point indicating a concentration of the target analyte in the patient at a particular time. The time series of data points may be presented on a display device of the continuous analyte monitoring system so the patient or caregiver can visualize the target analyte concentration in the patient over time.

In some instances, errors in the analyte sensor (e.g., errors caused by sensor chemistry, sensor compression, sensor temperature, etc.) cause gaps in the time series of data points. For example, sensor error may lead to certain analyte measurements being unreliable. The data points corresponding to these measurements may then be blanked or discarded (e.g., prevented from being displayed), which causes the time series to have gaps and to appear incomplete when displayed. Additionally, when the analyte concentrations are measured periodically (e.g., once every five minutes), the data points in the time series may be spaced apart from one another. As a result, the time series may appear noisy (e.g., jagged) when displayed. Due to the technical problems described above, existing analyte monitoring systems may generate sensor time series that may be or at least appear incomplete and/or noisy. The gaps and noise in the time series may cause confusion or stress for a patient or caregiver. For example, the gaps and noise may make it more challenging to interpret the time series and to understand whether remedial action is needed. If unresolved, the patient or caregiver may lose trust or confidence in the accuracy or quality of the continuous analyte monitoring system.

The present disclosure provides technical solutions that solve the technical problems described above. For example, the present disclosure describes a continuous analyte monitoring system that fills gaps in a time series of data points and that smooths the data points in the time series. For example, the system may use interpolation to determine data points used to fill gaps in the time series. Additionally, the system adjusts the values of data points in the time series with the average values of data points near or around the data points to reduce the noise in the time series and to make the time series appear smoother and less jagged when displayed. In this manner, the system transforms the time series of data points from the analyte sensor so that the time series includes fewer gaps and less noise.

In certain embodiments, the continuous analyte monitoring system described herein provides several technical advantages. For example, the system provides a continuous stream of data even when sensor errors occur. As another example, the system makes the times series easier to read and understand when displayed. Additionally, the system improves trust and confidence in the accuracy or quality of the continuous analyte monitoring system.

Additionally, for users with Type II diabetes who are insulin resistant, the analyte monitoring system may not generate data points as frequently relative to users who have other conditions. As a result, the time series of data points for a user with Type II diabetes may include larger time gaps with no data points and the time series of data points may appear noisy. The analyte monitoring system may fill in these gaps with data points and smooth the data points such that the analyte monitoring system provides the user with a time series that is easier to read and understand, which may also improve trust and confidence in the accuracy and quality of the analyte monitoring system.

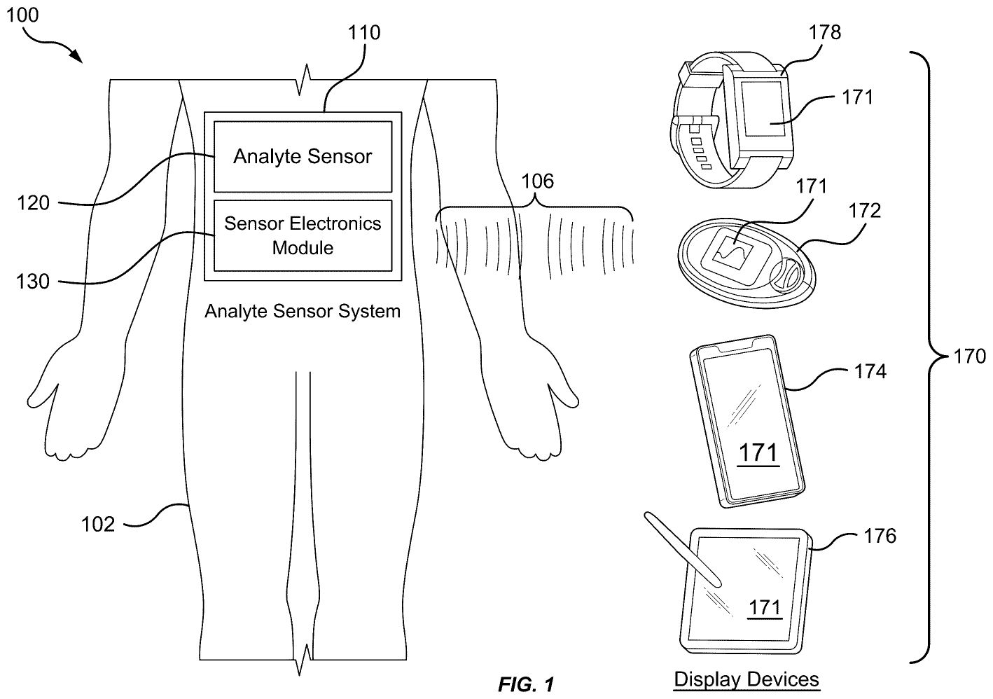

FIG. 1 illustrates an example continuous analyte monitoring system 100. As seen in FIG. 1, the system 100 includes an analyte sensor system 110 (e.g., positioned on a user 102) and display devices 170. Generally, the analyte sensor system 110 measures levels of analytes in the user 102 and communicates those measured levels to the display devices 170. In this manner, the continuous analyte monitoring system 100 assists the user 102 with decision support for managing a disease, e.g., diabetes, kidney disease, liver disease, or other types of discases.

The analyte sensor system 110 includes one or more continuous analyte sensors 120 (individually referred to herein as continuous analyte sensor 120 or analyte sensor 120 and collectively referred to herein as continuous analyte sensors 120 or analyte sensors 120) and a sensor electronics module 130. The sensor electronics module 130 may be in wired or wireless communication (e.g., directly or indirectly) with one or more of the display devices 170.

A continuous analyte sensor 120 may include one or more sensors for measuring analytes, including one or more multi-analyte sensors, each configured to continuously measure two or more analytes (e.g., glucose, lactate, potassium, ketone, etc.), and/or one or more single analyte sensors, each configured to continuously measure a single analyte. The continuous analyte sensor 120 may be a non-invasive device, a subcutaneous device, a transcutaneous device, a transdermal device, or an intravascular device. The continuous analyte sensor 120 may continuously measure analyte levels of a user using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The continuous analyte sensor 120 may provide a signal stream indicative of the concentration of one or more analytes in the user over time.

An analyte may be a substance or chemical constituent in a biological fluid (for example, blood, interstitial fluid, cerebral spinal fluid, lymph fluid, sweat, or urine) that can be analyzed. Analytes can include naturally occurring substances, artificial substances, metabolites, or reaction products. Analytes for measurement by the devices and methods may include, but may not be limited to, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycerol; glycosylated hemoglobin; halofantrine; hemoglobin variants; hexosaminidase A; human erythrocyte carbonic I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; potassium, quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, Leptospira,measles/mumps/rubella, Mycobacterium leprae, Mycoplasma pneumoniac, Myoglobin, Onchocerca volvulus, parainfluenza virus, Plasmodium falciparum, poliovirus, Pseudomonas acruginosa, respiratory syncytial virus, Rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

The continuous analyte sensor(s) 120 may include a percutaneous wire that has a proximal portion coupled to the sensor electronics module 130 and a distal portion with several electrodes, such as a measurement electrode and a reference electrode. The measurement (or working) electrode may be coated, covered, treated, embedded, etc., with one or more chemical molecules that react with a particular analyte, and the reference electrode may provide a reference electrical voltage. The measurement electrode may generate the analog electrical signal, which is conveyed along a conductor that extends from the measurement electrode to the proximal portion of the percutaneous wire that is coupled to the sensor electronics module 130. After the continuous analyte monitoring system 110 has been applied to the epidermis of the patient, the continuous analyte sensor(s) 120 penetrates the epidermis, and the distal portion extends into the dermis and/or subcutaneous tissue under epidermis. Other configurations of the continuous analyte sensor(s) 120 may also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, the continuous analyte sensor 120 may include a single-analyte sensor that measures lactate concentration levels, and another single-analyte sensor that measures glucose concentration levels of the patient. As another illustrative example, the continuous analyte sensor(s) 120 may include a single-analyte sensor that measures lactate concentration levels, and one or more multi-analyte sensors that measure glucose concentration levels, ketone concentration levels, creatinine concentration levels, etc. As yet another illustrative example, the continuous analyte sensor(s) 120 may include a multi-analyte sensor that measure lactate concentration levels, glucose concentration levels, ketone concentration levels, creatinine concentration levels, etc. Accordingly, the continuous analyte sensor(s) 120 generates at least one analog electrical signal that is proportional to the concentration level of a particular analyte, and sensor electronics module 130 converts the analog electrical signal into an analyte sensor count values, calibrate the analyte sensor count values based on the sensitivity profile of the continuous analyte sensor(s) 120 to generate measured analyte concentration levels, and transmit the measured analyte concentration level data, including the measured analyte concentration levels, to a display 170 via a wireless connection. For example, the sensor electronics module 130 may sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which may be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, the continuous analyte sensor(s) 120 may incorporate a thermocouple within, or alongside, the percutaneous wire to provide an analog temperature signal to the sensor electronics module 130, which may be used to correct the analog electrical signal or the measured analyte data for temperature. In other embodiments, the thermocouple may be incorporated into the sensor electronics module 130 above the adhesive pad, or, alternatively, the thermocouple may contact the epidermis of the patient through openings in the adhesive pad.

The sensor electronics module 130 includes electronic circuitry for measuring, processing, and adjusting signal streams from the analyte sensors 120 (which may be referred to as sensor data). The sensor electronics module 130 can be physically connected to the analyte sensors 120 and can be integral with (non-releasably attached to) or releasably attachable to the analyte sensors 120. The sensor electronics module 130 may include hardware, firmware, or software that enable measurement of levels of analytes via the analyte sensors 120. For example, the sensor electronics module 130 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, or a processor.

The display devices 170 may display sensor data, including measured levels of analytes or adjusted levels of analytes, which may be transmitted by the sensor electronics module 130. The sensor electronics module 130 may transmit raw sensor data that is converted to displayable sensor data via one or more of the display devices 170. The sensor electronics module 130 may convert raw sensor data to displayable sensor data and transmit the displayable sensor data to one or more of the display devices 170. Each of the display devices 170 may include a display such as a touchscreen display 171 for displaying sensor data to a user or for receiving inputs from the user. For example, a graphical user interface (GUI) may be presented to the user for such purposes. The display devices 170 may include other types of user interfaces such as a voice user interface instead of, or in addition to, a touchscreen display for communicating sensor data to the user of the display device or for receiving user inputs. The display devices 170 may display or otherwise communicate the sensor data as it is communicated from the sensor electronics module 130 (e.g., in a customized data package that is transmitted to the display devices 170 based on their respective preferences).

The display device 172 may include a custom display device specially designed for displaying certain types of displayable sensor data for analyte data received from the sensor electronics module 130. The display device 174 may be a smartphone or a mobile phone using a commercially available operating system (OS) and may display a graphical representation of the continuous sensor data (e.g., including current and historic data). The display device 176 may include a tablet, and the display device 178 may include a smart watch. The display devices 170 may include a desktop or laptop computer (not shown).

Because different display devices 170 provide different user interfaces, content of the data packages (e.g., amount, format, or type of data to be displayed, alarms, and the like) can be customized (e.g., programmed differently by the manufacture or by an end user) for each particular display device 170. Accordingly, different display devices 170 can be in direct wireless communication with the sensor electronics module 130 (e.g., such as an on-skin sensor electronics module 130 that is physically connected to the analyte sensors 120) during a sensor session to enable a plurality of different types or levels of display or functionality for the displayable sensor information.

The analyte sensor system 110 and the display devices 170 may communicate wireless signals 106 to each other using a variety of wireless communication technologies (e.g., Wi-Fi, Bluetooth, Near Field Communication (NFC), cellular, etc.). In some embodiments, a wireless access point (WAP) may be used to communicatively couple the analyte sensor system 110 and the display devices 170 to one another. For example, the WAP may provide Wi-Fi, Bluetooth, or cellular connectivity among these devices. NFC may also be used among the devices.

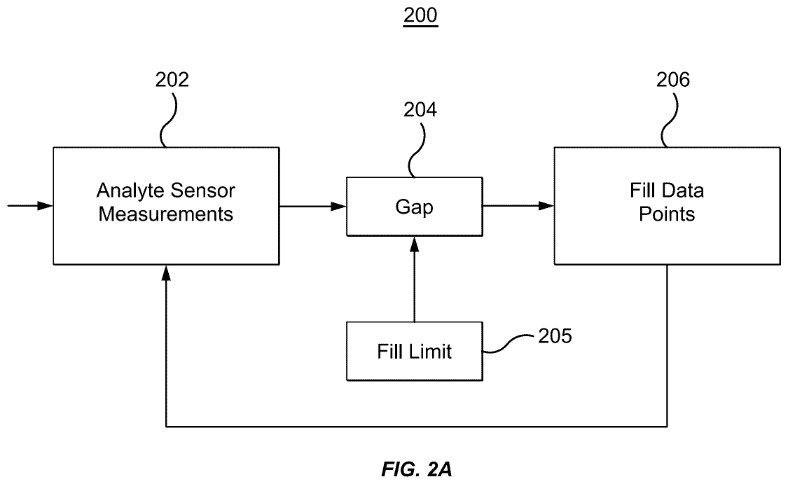

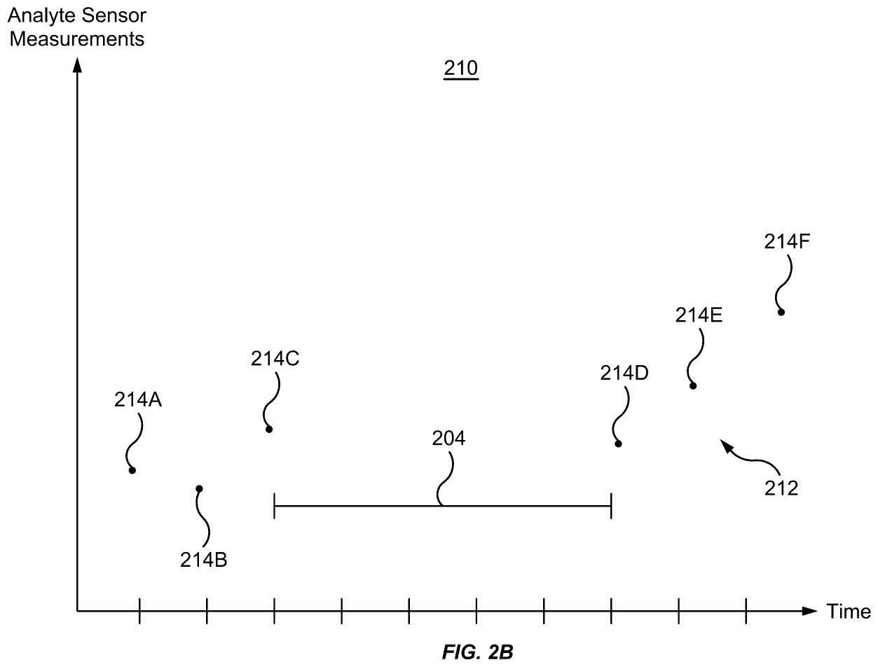

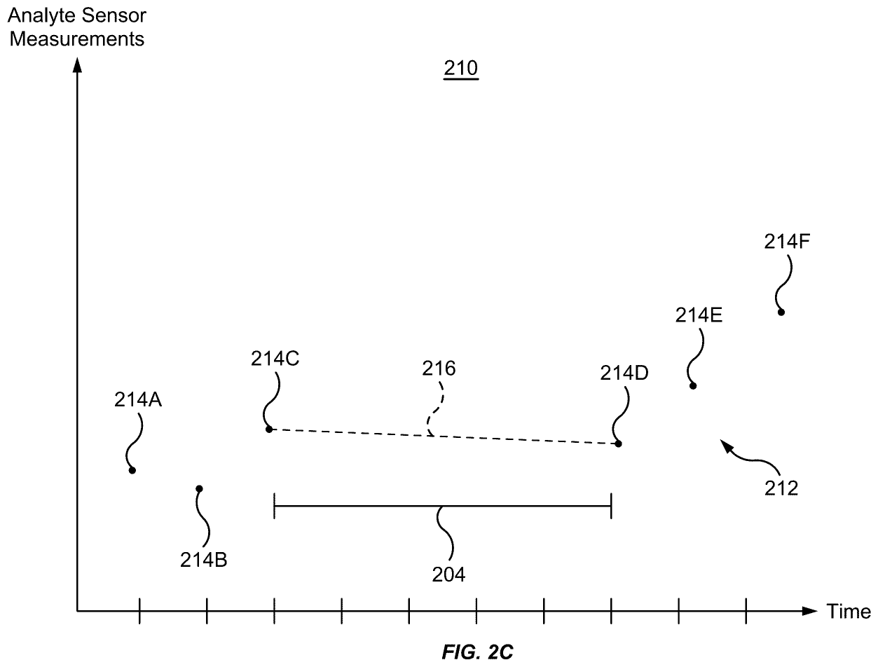

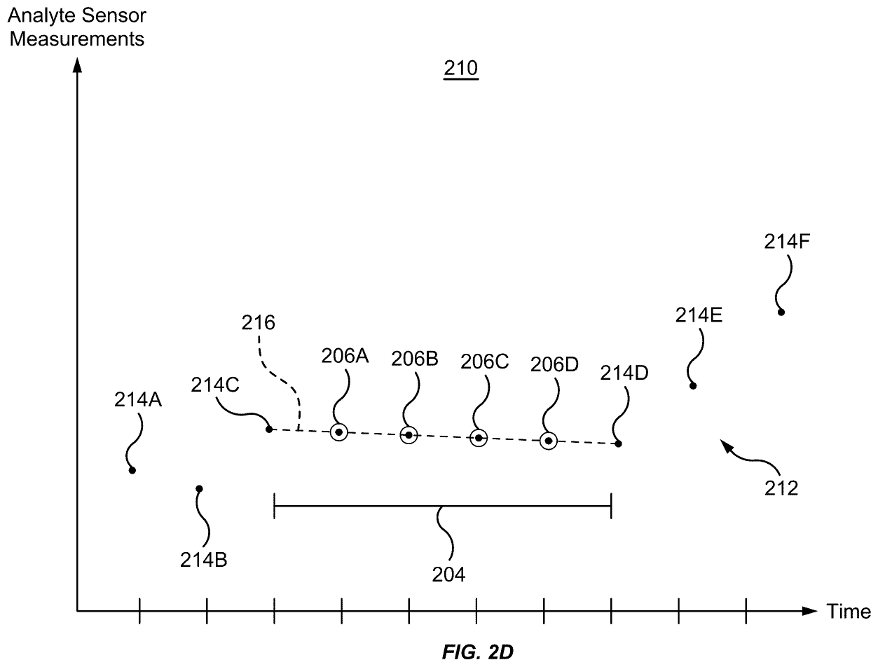

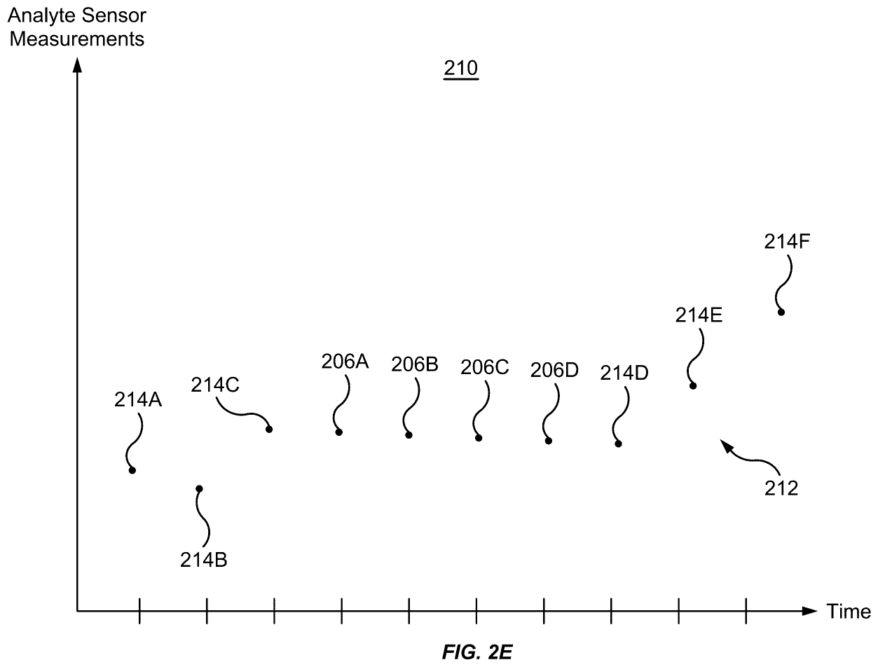

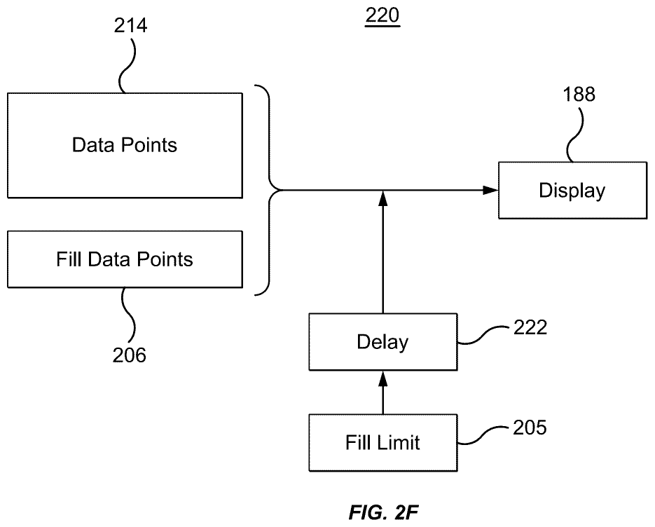

FIGS. 2A through 2F illustrate example operations for filling gaps in signal streams performed by the continuous analyte monitoring system 100 of FIG. 1, in accordance with certain aspects of the present disclosure. Generally, the display device (e.g., the display device 170 shown in FIG. 1) performs the operations shown in FIGS. 2A through 2F. By performing these operations, the display device detects and fills gaps in time series of data points from an analyte sensor system (e.g., the analyte sensor system 110 shown in FIG. 1).

FIG. 2A shows an example operation 200 performed by the display device. The display device begins by receiving analyte sensor measurements 202 from the analyte sensor system. The analyte sensor system may produce an electric signal depending on the concentration of a target analyte in a patient. The analyte sensor system then converts the electric signal into a measured analyte concentration value (e.g., an estimated glucose value) and communicates the analyte concentration value to the display device. The analyte sensor system may measure the analyte concentration periodically (e.g., every five minutes). The analyte sensor measurements 202 includes a time series of data points indicating the measured analyte concentration values at different times.

In some instances, errors may occur in the analyte sensor system that render the analyte concentration measurements unreliable. For example, compression of an analyte sensor of the analyte sensor system, sensor chemistry, or sensor temperature may cause the analyte sensor system to produce an inaccurate or unreliable analyte concentration value. In these instances, the analyte sensor system and/or the display device may blank or discard the data point in the time series that indicates the inaccurate or unreliable analyte concentration value to prevent the data point from being displayed and providing inaccurate or unreliable information to the patient. As a result, the analyte sensor measurements 202 include a gap 204 where the data point was blanked or discarded. When successive data points are blanked or discarded, the gap 204 is larger and spans a longer time duration.

The display device analyzes the analyte sensor measurements 202 to detect the gap 204 in the time series of data points. The display device may determine whether to fill the gap 204 using a fill limit 205. The fill limit 205 is a threshold that the display device uses to determine whether the gap 204 is too large and should not be filled. For example, the fill limit 205 may indicate a maximum size of the gap 204 (e.g., a maximum time duration or maximum amount of time covered by the gap 204) that the display device may fill. If the gap 204 is larger than the fill limit 205, the display device may not fill the gap 204. As another example, the fill limit 205 may indicate a maximum number of fill data points 206 that the display device may use to fill the gap 204. If the number of fill data points 206 needed to fill the gap 204 exceeds the fill limit 205, then the display device may not fill the gap 204. In this manner, the fill limit 205 prevents the display device from filling too large of a gap 204 and from using too many fill data points 206 to fill a gap 204, which preserves the accuracy and integrity of the time series of data points.

As an example, if the analyte sensor system measured the analyte concentration every five minutes and the gap 204 spanned 25 minutes, then four fill data points 206 would be used to fill the gap 204. If the fill limit 205 is 20 minutes or three fill data points 206, then the display device 107 may refrain from filling the gap 204.

If the display device determines that the fill limit 205 would not be exceeded, the display device determines the fill data points 206 to fill the gap 204. Generally, the display device performs interpolation to determine the values of the fill data points 206. For example, the display device may perform linear interpolation between the data points in the time series of data points on the edges of the gap 204 to determine the fill data points 206. As a result, the fill data points 206 fall on a line segment connecting the data points on the edges of the gap 204. The fill data points 206 may be spaced according to how frequently the analyte sensor system measures the analyte concentration. For example, if the analyte sensor system measures the analyte concentration every five minutes, then the fill data points 206 are spaced five minutes apart, consistent with the other data points in the time series of the analyte sensor measurements 202. In this manner, the display device uses the fill data points 206 to fill the gap 204 and potentially complete the time series of data points.

The display device may detect and fill any number of gaps 204 in the time series of data points. Additionally, the display device may detect any number of gaps 204 in the time series that exceed the fill limit 205 and leave those gaps 204 unfilled.

FIGS. 2B through 2E illustrate an example operation 210 performed by the display device to fill a gap 204. FIG. 2B shows a graph of analyte sensor measurements against time. As seen in FIG. 2B, the graph plots a time series 212 of data points 214 representing measured analyte concentration values. The graph includes the data points 214A, 214B, 214C, 214D, 214E, and 214F. Each data point 214A, 214B, 214C, 214D, 214E, and 214F indicates an analyte concentration measured at a particular time. As seen in FIG. 2B, the analyte concentration is measured periodically. For example, if the analyte concentration is measured every five minutes, then the data points 214A and 214B are spaced five minutes apart and the data points 214B and 2124C are spaced five minutes apart. Additionally, the data points 214D and 214E are spaced five minutes apart, and the data points 214E and 214F are spaced five minutes apart.

Additionally, the graph shows a gap 204 between the data points 214C and 214D. There may be no data points 214 in the gap 204 because the data points 214 have been blanked or discarded due to sensor error. As a result, the gap 204 causes the time series 212 to appear incomplete and unreliable. The display device may analyze the time series 212 and detect the gap 204. If the analyte concentration is measured every five minutes, then the gap 204 in the example of FIG. 2B spans twenty-five minutes.

As seen in FIG. 2C, the display device performs linear interpolation using the data points 214C and 214D on the edges of the gap 204 to determine a line segment 216 connecting the data points 214C and 214D. During linear interpolation, the display device may determine a mathematical function that describes the line segment 216 connecting the data points 214C and 214D. For example, the mathematical function may be a function of time and may specify a slope and intercept of the line segment 216.

As seen in FIG. 2D, the display device determines fill data points 206 to fill the gap 204. In the example of FIG. 2D, the display device determines the fill data points 206A, 206B, 206C, and 206D. Each of the fill data points 206A, 206B, 206C, and 206D fall on the line segment 216 connecting the data points 214C and 214D. The fill data points 206A, 206B, 206C, and 206D are also spaced apart in time with the same spacing as the data points 214. The display device may determine the fill data points 206A, 206B, 206C, and 206D by inputting the time values for blanked or discarded data points 214 in the time series 212 into the mathematical function for the line segment 216. The function would then output or produce the value of the fill data points 206A, 206B, 206C, and 206D at those time values.

As seen in FIG. 2E, the display device then adds the fill data points 206A, 206B, 206C, and 206D to the time series 212. In this manner, the display device fills the gap in the time series 212 and makes the time series 212 appear complete when displayed.

FIGS. 2F illustrates an example operation 220 performed by the display device to control the timing of when data points and fill data points are displayed on the display device. Generally, the display device may delay displaying fill data points to account for the fill limit 205.

The display device communicates the data points 214 and the fill data points 206 to the display 188 of the display device to display the data points 214 and the fill data points 206. The display device determines and applies a delay 222 to the communication of the fill data points 206 to the display 188. As a result, the display device may delay when fill data points 206 are displayed on the display device.

The display device may determine the delay 222 using the fill limit 205. For example, if the fill limit 205 indicates a duration of time (e.g., the maximum duration of a gap), then the delay 222 may be equal to the fill limit 205. As another example, if the fill limit 205 indicates a number of fill data points 206 (e.g., a maximum number of fill data points 206 that can be used to fill a gap), then the display device 170 may calculate the delay 222 by adding one to the fill limit 205 and multiplying by how regularly the analyte sensor system measures the analyte concentration (e.g., the amount of time between data points 214).

The display device applies the delay 222 to the fill data points 206. As a result, the display device may delay communicating the fill data points 206 for a particular gap until all the fill data points 206 for the gap are determined. For example, if the fill limit 205 is twenty-five minutes, then the display device may delay communicating fill data points 206 for a gap for twenty-five minutes. In this manner, the display device ensures that the fill data points 206 for the largest gap that the display device can fill according to the fill limit 205 are available before communicating the fill data points 206 to the display 188. For gaps that exceed the fill limit 205, the display device does not fill these gaps and does not communicate fill data points 206 to the display 188 for these gaps.

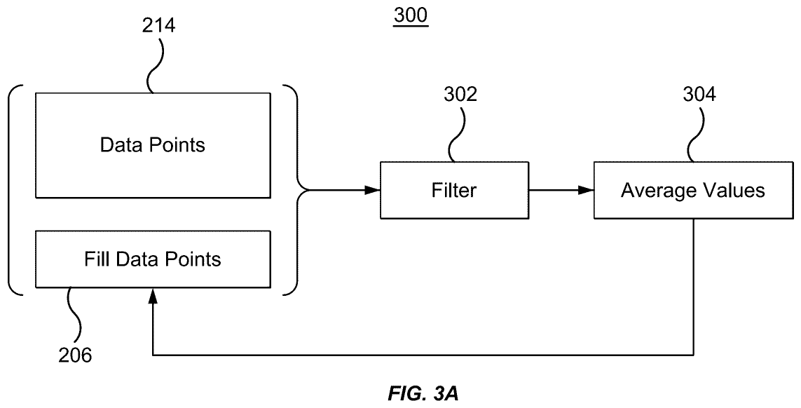

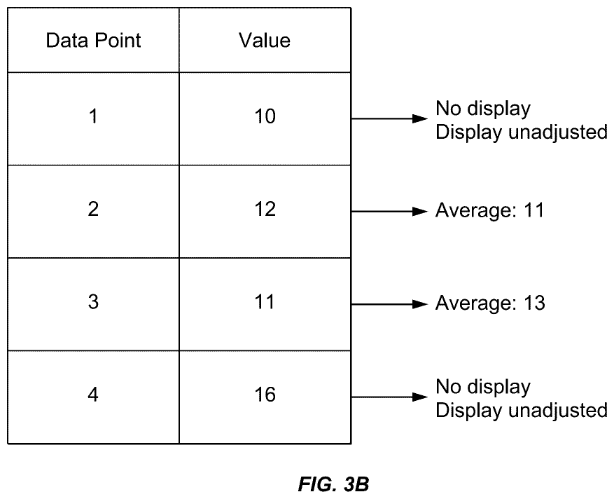

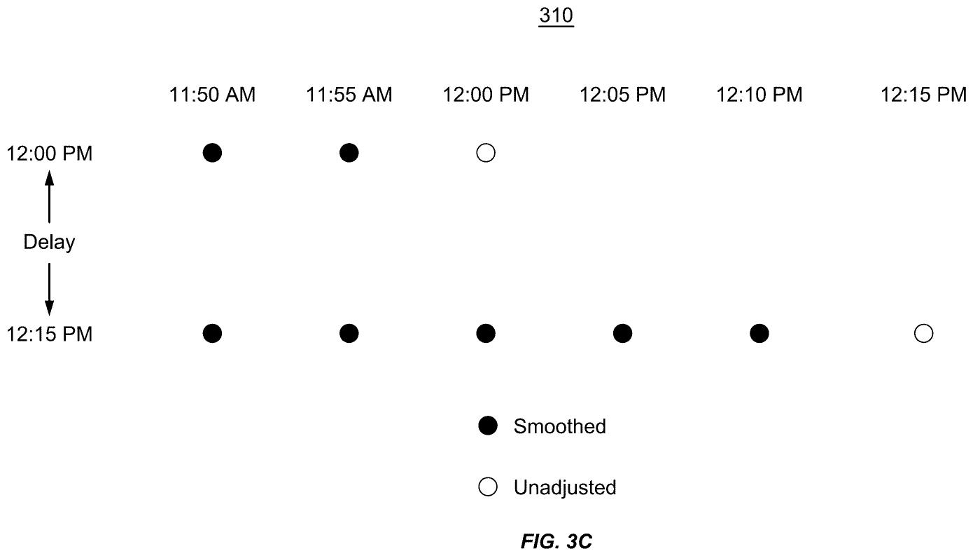

FIGS. 3A through 3C illustrate example operations for adjusting data points in signal streams performed by the continuous analyte monitoring system 100 of FIG. 1, in accordance with certain aspects of the present disclosure. Generally, a display device (e.g., the display device 170 shown in FIG. 1) performs the operations to reduce noise or the jagged appearance of a time series of data points.

FIGS. 3A shows an example operation 300 performed by the display device. The display device begins with the data points 214 that indicate analyte concentration values measured by an analyte sensor system (e.g., the analyte sensor system 110 of FIG. 1). The data points 214 may form a time series. Due to the analyte sensor system measuring analyte concentration values at regular intervals (e.g., every five minutes), the time series of data points 214 may appear noisy (e.g., jagged in appearance), which may give the impression that the time series is inaccurate or unreliable.

The display device adjusts the values of the data points 214 to reduce the noise in the time series and to smooth the time series. The display device may use a filter 302 to smooth the data points 214. For example, the filter 302 may be a moving average N-point filter. N could be an odd integer such as three, five, seven, etc. When the filter 302 is applied to a particular data point 214, the filter 302 calculates the average value of the N data points 214 in the time series centered on the particular data point. For example, if N is three, then the filter 302 calculates the average value of the particular data point 214, the data point 214 preceding the particular data point 214 in the time series, and the data point 214 following the particular data point 214 in the time series. Applying the filter 302 to the data points 214 in the time series produces average values 304 for the data points 214. The display device then adjusts or sets the values of the data points 214 in the time series to be their corresponding average values 304. By replacing the values of the data points 214 with the average values 304 from the filter 302, the display device reduces the jagged appearance of the time series, which may give the impression that the time series is reliable and accurate.

The display device may use the same technique when fill data points 206 are included in the time series. The display device may have determined the fill data points 206 to fill gaps in the time series. The display device may treat the fill data points 206 the same as the data points 214 when applying the filter 302. The display device may determine average values 304 for the fill data points 206 using the filter 302, and the display device may adjust or set the values of the fill data points 206 to their corresponding average values 304. Additionally, the filter 302 may use the values of the fill data points 206 when calculating the average values 304 of the data points 214 or fill data points 206.

Using the example shown in FIG. 2E, the display device may apply the filter 302 to the fill data points 206A, 206B, 206C, 206D, and 206E. For example, the filter 302 may calculate the average value 304 of the data point 214B, the data point 214C, and the fill data point 206A. The display device may then set the value of the data point 214C to that average value 304. As another example, the filter 302 may calculate the average value 304 of the fill data point 206A, the fill data point 206B, and the fill data point 206C. The display device may then set the value of the fill data point 206B to that average value 304. In this manner, the display device smooths the appearance of the time series 212 shown in FIG. 2E.

FIG. 3B shows the display device applying the filter to different data points. As seen in FIG. 3B, data point 1 may have a value of 10. Data point 2 may have a value of 12. Data point 3 may have a value of 11. Data point 4 may have a value of 16. The display device uses the filter to determine an average value of 11 for the data point 1, data point 2, and data point 3. The display device then sets the value of the data point 2 to 11. Additionally, the display device uses the filter to determine an average value of 13 for the data 2, data point 3, and data point 4. The display device then sets the value of the data point 3 to 13.

A moving average N-point filter may not correctly calculate an average value for data points that are on an edge, because these data points may not have data points that immediately precede and/or follow the data points. For example, the first data point in the time series may not have a preceding data point, and the final data point in the time series may not have a succeeding data point. As another example, data points on the edges of a gap may also not have a preceding data point or a succeeding data point. As a result, the moving average N-point filter does not have complete information from which to calculate the moving average for these data points. The display device may treat these types of data points differently.

In the example of FIG. 3B, data point 1 and data point 4 are on an edge. For example, these data points may be the first and last data points in the time series. As another example, one or more of these data points may be on the edge of a gap. No data point immediately precedes data point 1, and no data point immediately follows data point 4 in the time series. The display device may treat data point 1 and data point 4 differently from data point 2 and data point 3. For example, the display device may hide data point 1 and data point 4 from being displayed. As another example, the display device may display data point 1 and data point 4 using their unadjusted values (e.g., data point 1 with the value 10 and data point 4 with the value 16) rather than calculating a moving average for these data points and adjusting their values to the moving averages.

FIG. 3C illustrates an example operation 310 performed by the display device. Generally, the display device may delay when some data points are displayed to allow smoothing to occur. Additionally, the display device may retroactively adjust the values of some data points after these data points are displayed.

As seen in FIG. 3C, the display device receives an analyte concentration measurement every five minutes, and the display device presents three data points corresponding to the three most recent analyte concentration measurements every fifteen minutes. The display device displays three data points at 12:00 PM. The first data point corresponds to a measured analyte concentration at 11:50 AM. The second data point corresponds to a measured analyte concentration at 11:55 AM. The third data point corresponds to a measured analyte concentration at 12:00 PM. The data points at 11:50 AM and 11:55 AM have been adjusted to their corresponding moving average values. The data point at 12:00 PM is presented with an unadjusted value because at 12:00 PM, the data point at 12:05 PM is not yet available. As a result, the display device 170 may not be able to calculate the moving average corresponding to the data point at 12:00 PM yet.

Although data points may become available at 12:05 PM and 12:10 PM, the display device delays displaying these data points for fifteen minutes. At 12:15 PM, the display device displays the data points at 12:05 PM, 12:10 PM, and 12:15 PM. The data points at 12:05 PM and 12:10 PM have been adjusted to their corresponding moving average values. The data point at 12:15 PM is presented with an unadjusted value because at 12:15 PM, the data point at 12:20 PM is not yet available. As a result, the display device may not be able to calculate the moving average corresponding to the data point at 12:15 PM yet. Additionally, the display device retroactively adjusts the value of the data point at 12:00 PM to its corresponding moving average value. The display device can calculate the moving average value because the data point at 12:05 PM has become available.

The display device may determine the amount of delay (e.g., fifteen minutes in the example of FIG. 3C) before displaying the data points according to how many data points are considered by the moving average N-point filter (e.g., the size of N). In the example of FIG. 3C, because N is three and because analyte concentration is measured every five minutes, the delay is fifteen minutes (e.g., the product of three and five). Stated differently, because the filter needs fifteen minutes of information to calculate an average value for a data point, the display device delays for fifteen minutes for each group of data points.

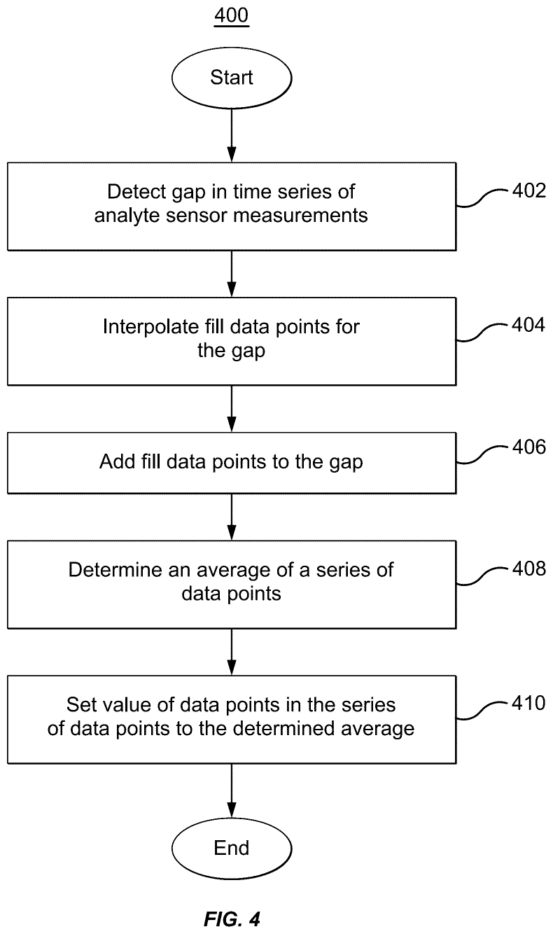

FIG. 4 is a flowchart of an example method 400 performed by the continuous analyte monitoring system 100 of FIG. 1, in accordance with certain aspects of the present disclosure. In certain embodiments, a display device (e.g., the display device 170 shown in FIG. 1) performs the method 400. By performing the method 400, the display device fills gaps in a time series of data points and smooths the data points in the time series.

At 402, the display device detects a gap in a time series of analyte sensor measurements. The time series may include data points, with each data point representing an analyte concentration measurement made by an analyte sensor system (e.g., the analyte sensor system 110 shown in FIG. 1). The data points in the time series may be spaced at regular time intervals consistent with how frequently the analyte sensor system measures analyte concentration levels. The gap in the time series may result from sensor errors in the analyte sensor system. The analyte sensor system and/or the display device may blank or discard data points when a sensor error is detected. Blanking or discarding the data points creates the gap in the time series.

At 404, the display device interpolates fill data points for the gap. For example, the display device may perform linear interpolation using the two data points at the edges of the gap to determine a line segment connecting the two data points. The fill data points may fall on this line segment. At 406, the display device adds the fill data points to the gap. The fill data points may be spaced from each other and from other data points in the time series at the same regular time intervals consistent with how frequently the analyte sensor system measures analyte concentration levels. In this manner, the display device fills the gap in the time series, which may cause the time series to appear complete.

At 408, the display device determines an average of a series of data points in the time series. For example, the display device may use a moving average N-point filter that calculates a moving average for a data point by averaging the values of N data points centered on that data point. At 410, the display device sets the value of that data point to the determined moving average for that data point. The display device may repeat this process to adjust the values of multiple data points in the time series. In this manner, the display device smooths the appearance of the time series when displayed.

In summary, the continuous analyte monitoring system 100 fills gaps in a time series of data points and smooths the data points in the time series. For example, the system 100 may use interpolation to determine data points used to fill gaps in the time series. Additionally, the system 100 adjusts the values of data points in the time series with the average values of data points near or around the data points to reduce the noise in the time series and to make the time series appear smoother and less jagged when displayed. In this manner, the system 100 transforms the time series of data points from the analyte sensor so that the time series includes fewer gaps and less noise.

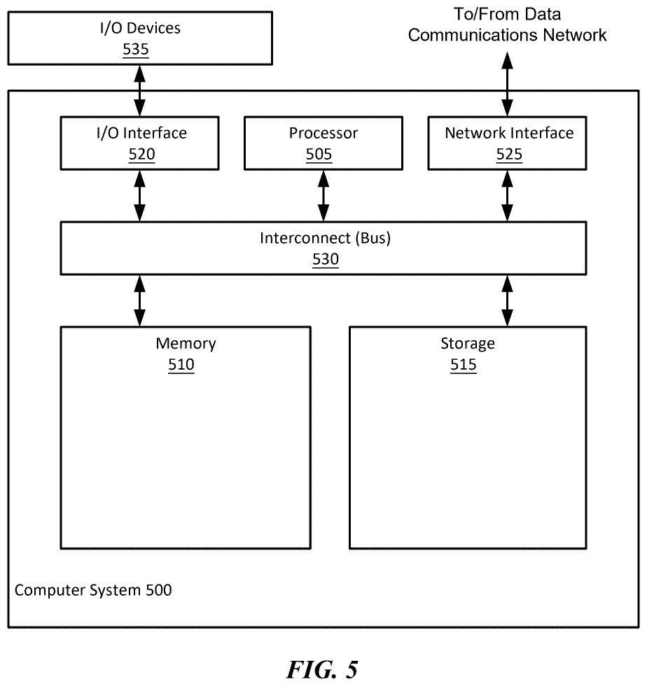

FIG. 5 is a block diagram depicting a computer system 500, which may be the display device 170 shown in FIG. 1. Although depicted as a single physical device, in embodiments, the computer system 500 may be implemented using virtual device(s), and/or across a number of devices, such as in a cloud environment and/or via separate modules of portable or cloud devices. As illustrated, the computer system 500 includes a processor 505, a memory 510, a storage 515, a network interface 525, and one or more I/O interfaces 520. In the illustrated embodiment, the processor 505 retrieves and executes programming instructions stored in the memory 510, as well as stores and retrieves application data residing in the storage 515. The processor 505 is generally representative of a single CPU and/or GPU, multiple CPUs and/or GPUs, a single CPU and/or GPU having multiple processing cores, and the like.

The processor 505 is any electronic circuitry, including, but not limited to one or a combination of microprocessors, microcontrollers, application specific integrated circuits (ASIC), application specific instruction set processor (ASIP), and/or state machines, that communicatively couples to the memory 510 and controls the operation of the computer system 500. The processor 505 may be 8-bit, 16-bit, 32-bit, 64-bit or of any other suitable architecture. The processor 505 may include an arithmetic logic unit (ALU) for performing arithmetic and logic operations, processor registers that supply operands to the ALU and store the results of ALU operations, and a control unit that fetches instructions from memory and executes them by directing the coordinated operations of the ALU, registers and other components. The processor 505 may include other hardware that operates software to control and process information. The processor 505 executes software stored on the memory 510 to perform any of the functions described herein. The processor 505 controls the operation and administration of the computer system 500 by processing information (e.g., information received from the memory 510). The processor 505 is not limited to a single processing device and may encompass multiple processing devices contained in the same device or computer or distributed across multiple devices or computers. The processor 505 is considered to perform a set of functions or actions if the multiple processing devices collectively perform the set of functions or actions, even if different processing devices perform different functions or actions in the set.

The memory 510 is generally included to be representative of a random access memory (RAM). The storage 515 may be any combination of disk drives, flash-based storage devices, and the like, and may include fixed and/or removable storage devices, such as fixed disk drives, removable memory cards, caches, optical storage, network attached storage (NAS), or storage area networks (SAN). The memory 510 may store, either permanently or temporarily, data, operational software, or other information for the processor 505. The memory 510 may include any one or a combination of volatile or non-volatile local or remote devices suitable for storing information. For example, the memory 510 may include random access memory (RAM), read only memory (ROM), magnetic storage devices, optical storage devices, or any other suitable information storage device or a combination of these devices. The software represents any suitable set of instructions, logic, or code embodied in a computer-readable storage medium. For example, the software may be embodied in the memory 510, a disk, a CD, or a flash drive. In particular embodiments, the software may include an application executable by the processor 505 to perform one or more of the functions described herein. The memory 510 is not limited to a single memory and may encompass multiple memories contained in the same device or computer or distributed across multiple devices or computers. The memory 510 is considered to store a set of data, operational software, or information if the multiple memories collectively store the set of data, operational software, or information, even if different memories store different portions of the data, operational software, or information in the set.

In some embodiments, the I/O devices 535 (such as keyboards, monitors, etc.) can be connected via the I/O interface(s) 520. Further, via the network interface 525, the computer system 500 can be communicatively coupled with one or more other devices and components. In certain embodiments, the computer system 500 is communicatively coupled with other devices via a network, which may include the Internet, local network(s), and the like. The network may include wired connections, wireless connections, or a combination of wired and wireless connections. As illustrated, the processor 505, memory 510, storage 515, network interface(s) 525, and the I/O interface(s) 520 are communicatively coupled by one or more interconnects 530. In certain embodiments, the computer system 500 is representative of the display device associated with the user. In certain embodiments, as discussed above, the display device can include the user's laptop, computer, smartphone, and the like. In another embodiment, the computer system 500 is a server executing in a cloud environment.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

The term “continuous,” as used herein, is a broad term, and is used in its ordinary sense, and can mean continuous, semi-continuous, continual, periodic, intermittent, regular, etc.

The terms “continuous analyte sensor,” “continuous multi-analyte sensor,” “continuous glucose sensor,” and “continuous lactate sensor,” as used herein, are broad terms, and are used in their ordinary sense, and refer without limitation to a device that continuously measures a concentration of an analyte or calibrates the device (e.g., by continuously adjusting or determining the sensor's sensitivity and background), for example, at time intervals ranging from fractions of a second up to, e.g., 1, 2, or 5 minutes, or longer.

The terms “sensitivity” or “sensor sensitivity,” as used herein, are broad terms, and are used in their ordinary sense, and refer without limitation to an amount of signal produced by a certain concentration of a measured analyte, or a measured species (e.g., H2O2) associated with a measured analyte (e.g., glucose or lactate). For example, a sensor may have a sensitivity of from about 1 to about 300 picoAmps of current for every 1 mg/dL of glucose analyte.

The term “sensor data,” as used herein, is a broad term, and is used in its ordinary sense, and refers without limitation to any data associated with a sensor, such as a continuous analyte or continuous multi-analyte sensor. Sensor data includes a raw data stream, or simply data stream, of analog or digital signal directly related to a measured analyte from an analyte sensor (or other signal received from another sensor), as well as calibrated or filtered raw data. The terms “sensor data point” and “data point” refer generally to a digital representation of sensor data at a particular time. The terms broadly encompass a plurality of time spaced data points from a sensor, such as a continuous analyte sensor, which comprises individual measurements taken at time intervals ranging from fractions of a second up to, e.g., 1, 2, or 5 minutes or longer. In another example, the sensor data includes an integrated digital value representative of one or more data points averaged over a time period. Sensor data may include calibrated data, smoothed data, filtered data, transformed data, or any other data associated with a sensor.

The term “sensor electronics,” as used herein, is a broad term, and is used in its ordinary sense, and refers without limitation to components, e.g., hardware or software, of a device configured to process sensor data.

Although certain embodiments herein are described with reference to management of diabetes, diabetes management is only an example of one application for which the present systems and methods may be utilized. The systems and methods described herein can also be used for managing one or more other diseases or conditions, which may or may not include diabetes. For example, the systems and methods described herein can be utilized for managing kidney disease, liver disease, and other types of diseases or conditions.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.”

While various examples have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the embodiments, but instead as merely intended to highlight alternative or additional features that may or may not be utilized in a particular example. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the disclosure to the specific examples and examples described herein, but rather to also cover all modification and alternatives coming with the true scope and spirit of the disclosure.

What is claimed is:

1. An analyte monitoring system comprising:
an analyte sensor system comprising:
an analyte sensor configured to generate a sensor current; and
sensor electronics configured to generate analyte sensor measurements based on the sensor current;
a memory; and
a processor communicatively coupled to the memory, the processor configured to:
detect a first gap in a time series of analyte sensor measurements from the analyte sensor system, wherein the first gap is between a first data point in the time series and a second data point in the time series;
interpolate between the first data point and the second data point to determine a fill data point;
add the fill data point to the first gap in the time series;
determine an average of at least (i) a value of the first data point, (ii) the fill data point, and (iii) a third data point preceding the first data point in the time series; and
set the value of the first data point to the determined average.

2. The analyte monitoring system of claim 1, wherein the fill data point is on a line segment connecting the first data point and the second data point.

3. The analyte monitoring system of claim 1, wherein the processor is configured to add a plurality of fill data points to the first gap based on a fill limit.

4. The analyte monitoring system of claim 1, wherein the processor is further configured to:
detect a second gap in the time series of analyte sensor measurements from the analyte sensor system; and
leave the second gap unfilled based on a fill limit.

5. The analyte monitoring system of claim 1, wherein the processor is further configured to delay displaying the fill data points based on a fill limit.

6. The analyte monitoring system of claim 1, wherein the processor is further configured to refrain from displaying a final data point in the time series of analyte sensor measurements from the analyte sensor system.

7. The analyte monitoring system of claim 1, wherein the processor is further configured to delay displaying the first data point based on a number of data points used to determine the average.

8. The analyte monitoring system of claim 1, wherein the processor is further configured to:
display the first data point prior to setting the value of the first data point to the average; and
adjust the displayed first data point after setting the value of the first data point to the average.

9. A method comprising:
detecting a first gap in a time series of analyte sensor measurements from an analyte sensor system, wherein the first gap is between a first data point in the time series and a second data point in the time series;
interpolating between the first data point and the second data point to determine a fill data point;
adding the fill data point to the first gap in the time series;
determining an average of at least (i) a value of the first data point, (ii) the fill data point, and (iii) a third data point preceding the first data point in the time series; and
setting the value of the first data point to the determined average.

10. The method of claim 9, wherein the fill data point is on a line segment connecting the first data point and the second data point.

11. The method of claim 9, further comprising adding a plurality of fill data points to the first gap based on a fill limit.

12. The method of claim 9, further comprising:
detecting a second gap in the time series of analyte sensor measurements from the analyte sensor system; and
leaving the second gap unfilled based on a fill limit.

13. The method of claim 9, further comprising delaying displaying the fill data points based on a fill limit.

14. The method of claim 9, further comprising refraining from displaying a final data point in the time series of analyte sensor measurements from the analyte sensor system.

15. The method of claim 9, further comprising delaying displaying the first data point based on a number of data points used to determine the average.

16. The method of claim 9, further comprising:
displaying the first data point prior to setting the value of the first data point to the average; and
adjusting the displayed first data point after setting the value of the first data point to the average.

17. A glucose monitoring system comprising:
an analyte sensor system configured to:
measure glucose concentration levels of a user; and
transmit a time series of data points indicating the glucose concentration levels of the user;
a memory; and
a processor communicatively coupled to the memory, the processor configured to:
detect a first gap in the time series, wherein the first gap is between a first data point in the time series and a second data point in the time series;
interpolate between the first data point and the second data point to determine a fill data point;
add the fill data point to the first gap in the time series;
determine an average of a plurality of data points in the time series; and
set a value of a data point of the plurality of data points to the determined average.

18. The glucose monitoring system of claim 17, wherein the processor is configured to add a plurality of fill data points to the first gap based on a fill limit.

19. The glucose monitoring system of claim 17, wherein the processor is further configured to:
detect a second gap in the time series of analyte sensor measurements from the analyte sensor system; and
leave the second gap unfilled based on a fill limit.

20. The glucose monitoring system of claim 17, wherein the processor is further configured to delay displaying the data point of the plurality of data points based on a number of data points in the plurality of data points.
